(19) United States
(12) Patent Application Publication
(10) Pub. No.: US 2013/0345757 A1
(43) Pub. Date: Dec. 26, 2013

(54) Image Guided Intra-Operative Contouring Aid

(71) Applicant: Shawn D. Stad, Fall River, MA (US)

(72) Inventor: Shawn D. Stad, Fall River, MA (US)

(21) Appl. No.: 13/530,441

(22) Filed: Jun. 22, 2012

(51) Int. Cl. A61B 17/88

(52) U.S. Cl. 606/279

(57) ABSTRACT

A method of contouring spinal rods, and systems therefor. The surgeon uses image guided surgery instruments to identify the locations of the screw heads through which the rod will pass. These locations allow a computer to form a best fit line that corresponds to the shape of a rod that can pass through the screw heads. This best fit line is then displayed on a projection table from both its coronal and sagittal views. The surgeon then shapes the rod using these 2-D images as a template.

### BACKGROUND OF THE INVENTION

Spine surgeries involving the correction of deformities or degenerative disc disease often utilize spinal rods as a means of placing the spinal column in a fixed position. These rods are used to connect the heads of pedicle screws that are placed in successive vertebrae in the spinal column around the region of deformity or degeneration. Because the spinal rod is often provided in a straight length, the surgeon must cut the rod to an appropriate length and then contour the rod to the appropriate spinal curvature.

Rod contouring in complex deformity cases is a highly specialized procedure. It requires the surgeon to possess spatial cognition and an ability to visualize the partially exposed spine in three dimensions. Typically, several adjustments are made to the rod during the contouring procedure. These adjustments add time to the overall procedure, thereby adding to the cost of the operation and the time the patient is under anesthesia. Intraoperative adjustment also increases the stress upon the rod.

These challenges described above are heightened during minimally invasive procedures, because the head of the polyaxial screw is not visible and the surgeon must pass the rod percutaneously.

Often, the surgeon will not adjust the rod, but instead use a powerful reduction instrument to force the rod into the screw head, thereby sacrificing optimal correction.

### SUMMARY OF THE INVENTION

The present invention relates to a method of contouring spinal rods, and systems therefor.

The surgeon uses image guided surgery instruments to identify the locations of the screw heads through which the rod will pass. These locations allow a computer to form a best fit line that corresponds to the shape of a rod that can pass through the screw heads. This best fit line is then displayed on a projection table from both its coronal and sagittal views. The surgeon then shapes the rod using these 2-D images as a template.

Therefore, in accordance with the present invention, there is provided a method comprising the steps of:
a. implanting a plurality of pedicle screws into the spine of a patient, each screw having a head,
b. coupling (preferably, attaching) a tracking device to each head to allow a computer system to construct a virtual rod therefrom,
c. reading a geometric descriptor of the virtual rod displayed by the computer system, and
d. cutting a length of a rod blank based upon the geometric descriptor of the virtual rod.

Also in accordance with the present invention, there is provided a method comprising the steps of:
a) identifying locations of a plurality of screw heads attached to the spine of a patient,
b) creating a virtual rod from the locations of the screw heads, and
c) communicating a geometric descriptor of the virtual rod.

Also in accordance with the present invention, there is provided a computer comprising:
a) means for identifying locations of a plurality of screw heads attached to the spine of a patient,
b) means for creating a virtual rod from the locations of the screw heads.

Also in accordance with the present invention, there is provided a method comprising the steps of:
a) implanting a plurality of implants (preferably, threaded implants) into the spine of a patient,
b) coupling (preferably attaching) a tracking device to each implant to allow a computer system to construct a virtual rod therefrom,
c) reading a geometric descriptor of the virtual rod displayed by the computer system.

### DESCRIPTION OF THE FIGURES

FIG. 1 is a coronal view of a scoliotic spine.

FIG. 2 is a coronal view of a scoliotic spine having a plurality of pedicle screws implanted therein.

FIG. 3 discloses the head locator instrument nested within a screw head that has been implanted into a scoliotic spine.

FIG. 4 discloses the relative positions of points identified by the Head locator instrument, wherein these points correspond to screw head locations.

FIG. 5 discloses a touch screen display of the present invention.

FIG. 6 discloses a projection system of the present invention.

FIG. 7 discloses the head locator instrument.

FIG. 8 discloses a computerized system of the present invention.

### DETAILED DESCRIPTION OF THE INVENTION

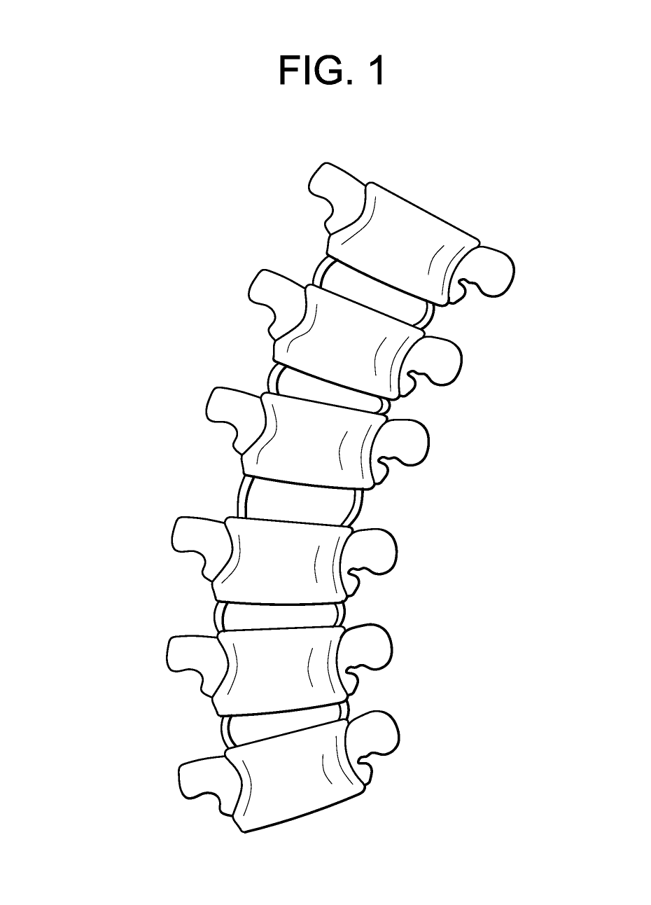

The methods of the present invention are preferably intended for use in scoliotic spines and in spines undergoing a fusion. One scoliotic spine, with its curved shape, is shown in FIG. 1.

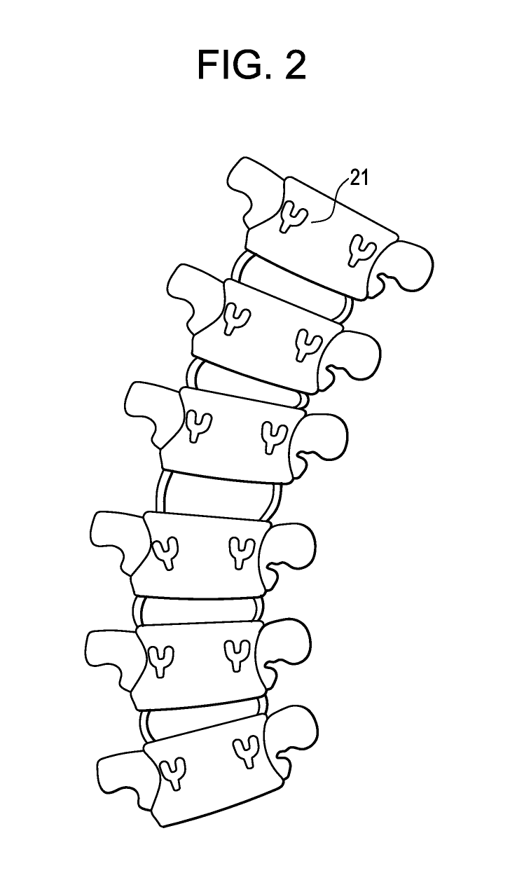

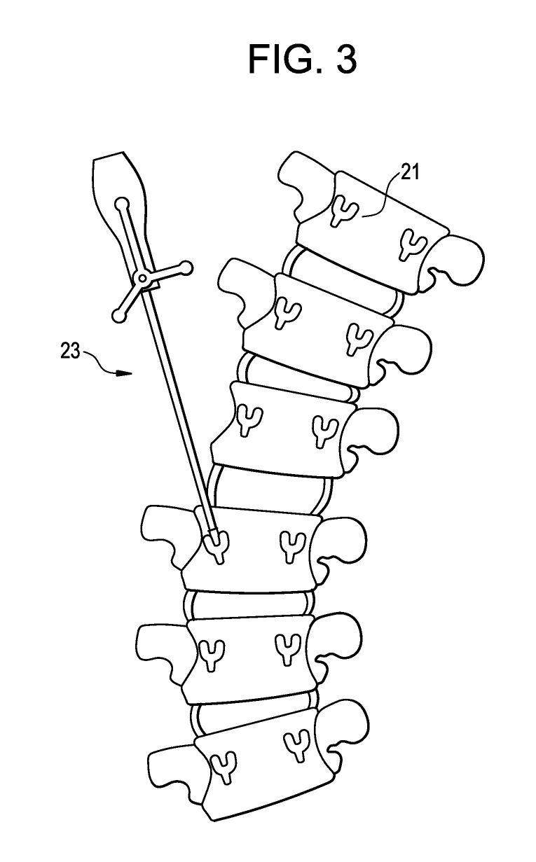

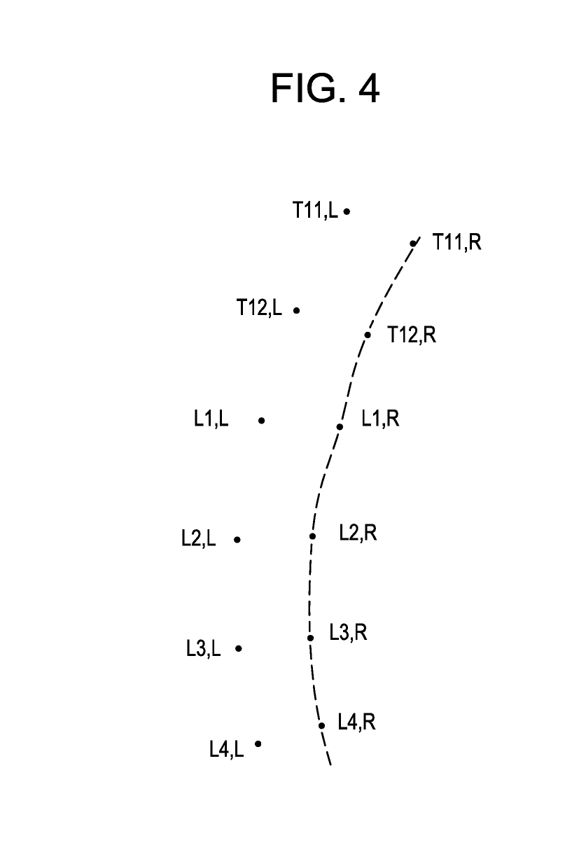

Now referring to FIG. 2, to begin the procedure, the surgeon inserts a plurality of pedicle screws into the spinal column of a patient so that the heads 21 of the screws extend outward from the vertebral bodies. Next, and now referring to FIG. 3, the surgeon places a distal tip of a tracking device 23 upon the apex of the receiving surface of the head of each inserted pedicle screw. The tracking device allows a computer to identify the location of the distal tip, and thereby identify the geometric center of each screw head in 3-dimensional space. Now referring to FIG. 4, the computer system then plots each of these centers in 3D space and generates a best fit line that corresponds to a contoured virtual rod. The length and shape of this virtual rod is optimized for the particular locations of the screw heads.

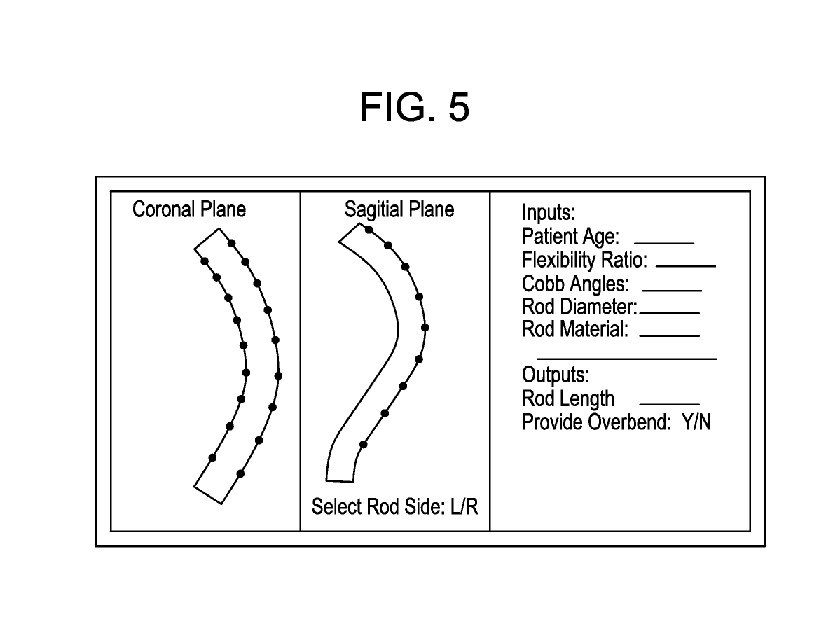

Optionally, the surgeon has the ability to adjust the virtual location of a screw head to accommodate for deformity correction and the desired final positioning of the screw heads. Now referring to FIG. 5, these alterations may be carried out by the surgeon by manipulating on a touch screen an image of the virtual rod superimposed over the patient's spinal column. These alterations produce an altered virtual rod.

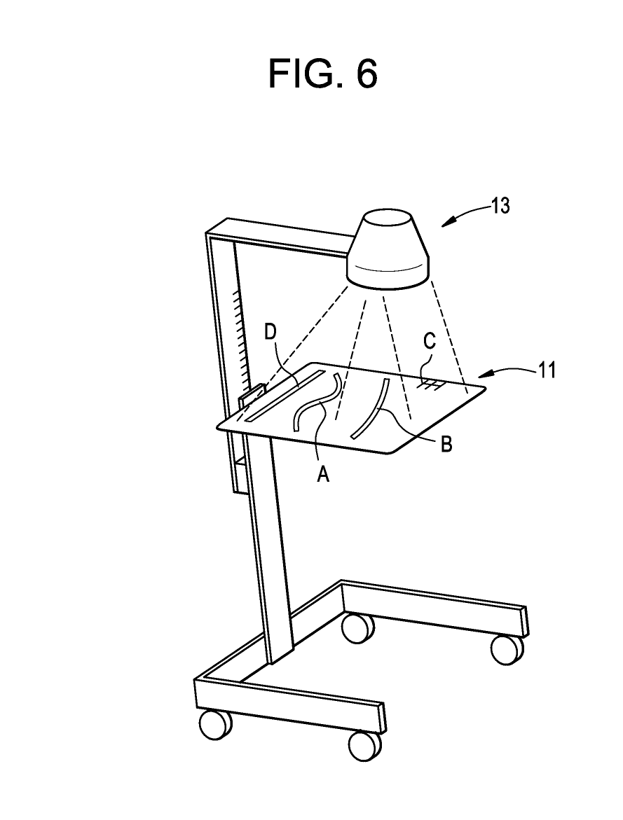

Now referring to FIG. 6, once the desired virtual rod contour is achieved, the computer system then projects an image of straight virtual rod onto a projection tray, wherein the straight rod has the same length of the virtual rod determined by the best fit line. The surgeon uses this image to cut a physical rod from a length of rod material (a “rod blank”) so that the physical rod has the same length as the virtual rod.

Once the surgeon cuts the appropriate length of rod, the computer system then projects precise contoured 2D images (e.g., in the sagittal and coronal planes) of the rod onto a projection surface at a known distance so that the rod images on the projection surface correspond exactly to the dimensions and curvature of the virtual rod. These surface images are then used as templates for the surgeon to contour a physical rod into a desired shape.

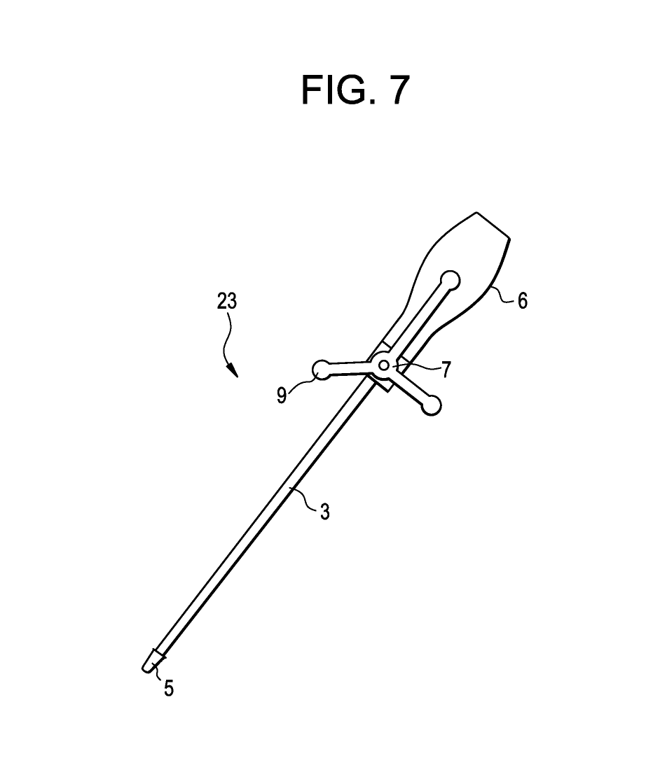

The head locator probe of the present invention can be tracked by a computer system so as to allow for the identification of its tip location by its coordinates in 3-dimensional space. Now referring to FIG. 7. In its simplest form, the head locator probe 23 comprises a rod 3 having a distal tip 5, a proximal handle 6, and an intermediate tracker 7. Generally, the tracker comprises a plurality of tracking means 9, preferably three tracking means, for generating a signal representing the trajectory of the tool and the depth of the instrument tip. Preferably, the tracking means are passive, and more preferably comprise reflective surfaces. However, other tracking devices known in the art and capable of being tracked by a corresponding sensor array are within the scope of the present invention. For the purposes of illustration, and not limitation, the head locator probe may generate signals actively such as with acoustic, magnetic, electromagnetic, radiologic and micropulsed systems, and emitters such as LEDs.

In some embodiments, the tracking means comprise light reflectors or light emitters.

For the purposes of the present invention, the “base length” is defined to be the length of the best fit line between the points represented by the uppermost and lowermost screw heads. Thus, the length of the virtual rod will include at least the base length. In some embodiments, a fixed length such as 2-3 mm will be added to each end of the base length to form the virtual rod. In other embodiments, a fixed percentage of the base length (such as 5% of the base length) will be added to each end of the base length to form the virtual rod. In some embodiments, the surgeon may want to add even more length to the base length of the virtual rod in order to provide adequate rod length for suitable connection to extend the construct should a secondary procedure be required.

After the virtual rod is virtually constructed, a geometric descriptor of its length is first communicated to the surgeon so that the surgeon may first cut a particular length of a physical rod blank to correspond with the length of the virtual rod. In some embodiments, the computer may simply communicate the length of the virtual rod in metric terms, such as in millimeters. In other some embodiments, the computer may communicate the length of the virtual rod by projecting onto a surface a 2D image of a straight rod having the same length as the virtual rod. Such a straight virtual rod is shown in FIG. 6 as image D. The surgeon can then lay the rod blank upon the image and cut the blank to the length of the virtual rod. In either case, a straight physical rod whose length corresponds with the length of the virtual rod is produced.

The projection surface of the present invention includes any substantially flat surface in the operating room onto which a visual 2D image may be accurately projected. In some preferred embodiments, the projection surface is derived from a Mayo stand. Now referring to FIG. 6, the stand may include a projection surface 11 and a projection lamp 13 which projects the images A-D onto the projection surface. In some embodiments, there is provided a means of finely adjusting the distance between the projector and the projection surface. There may be an actual marker (scale) on the projection table and then the projection height is adjusted until the actual scale and the virtual scale match. The same could automatically occur via the system during a calibration procedure in which the system adjusts the location of the projection surface or adjusts the image.

In some embodiments, the cut blank is laid upon the sagittal and coronal images of the contoured virtual rod (images A and B in FIG. 6) and this cut blank is then bent to correspond with images A and B and thereby produce the contoured physical rod. The contoured physical rod is then inserted into the pedicle screw heads that were used to construct the virtual rod.

In some embodiments, patient-specific parameters such as flexibility ratio may also be inputted into the computer system. The system may use the patient's particular flexibility ratio (which is the ratio of the curvature on the standing or supine film to that of the curvature as measured on flexion/extension films) to assess whether a particular virtual rod (which has a particular contour) is within the bounds of that patient's flexibility.

Another parameter that a surgeon can provide is the rod material. By knowing the rod material as well as the curvature of the best fit curve obtained from the screw head locations, the system could calculate and then provide the amount of over-contouring (or “overbending”) necessary for each rod. To explain further, surgeons typically overbend the concave side of the physical rod, understanding that the rod will flatten out to an extent intra- and post-operatively.

### EXAMPLE

The method of the present invention is generally carried out on a patient having a deformed spine, such as a patient having a scoliotic spine. One example of a scoliotic spine is provided in FIG. 1.

Now referring to FIG. 2, pedicle screws are placed bilaterally in the pedicles of the patient's spine. These screws can be placed via an MIS, mini-open or open approach.

Next, and now referring to FIG. 3, the distal end of the Head Locator instrument is contacted to the head of each pedicle screw. The distal end nests in the head of each screw to precisely identify the location where the central axis of a spinal rod passing through the screws would be located. With the help of the IGS computer system, the instrument identifies the location of each screw head for each side of the spine in the X, Y and Z planes.

Now referring to FIG. 4, the computer system creates a best fit curve from the points corresponding to screw head locations.

Now referring to FIG. 5, a touch screen can display the location of the points corresponding to the screw heads. Further, the screw heads (or their respective points) can also be shown at their locations on the spine by registering with a pre-operative or intra-operative CT. Although FIG. 5 shows the sagittal and coronal views of the virtual rod, the virtual rod could also be displayed via a 3D reconstruction that the surgeon could manipulate via the touch screen.

In some embodiments, the surgeon is able to manipulate the screw head points using the touch screen, thereby altering the virtual rod to meet the surgeon's requirements. If desired, the system can then assess parameters such as flexibility ratio and, if needed, indicate that the surgeon has moved a given point beyond the achievable range.

Providing rod-related information, such as diameter and material, enables the system to provide an appropriate amount of overbend. Surgeons overbend a rod because rod will tend to flatten out during reduction. This flattening is more likely to occur with less stiff materials such as titanium.

Now referring to FIG. 6, the virtual rod is displayed on a projection tray in the form of a sagittal projection image A, a coronal projection image B and a straight length image C. The straight length C image allows the surgeon to place a straight rod blank on the tray and cut a section of rod need to make a physical rod having the curves shown in images A and B. Ruler D provides a metric to insure that the projected images are displaying at the appropriate dimensions. In some embodiments, the surgeon could preload a temporary clamp on the rod that helps the surgeon to maintain orientation as the surgeon is contouring and when the surgeon sees the rod on the tray to check against the projected curves.

Preferably, the tools of the present invention are used in conjunction with a computer assisted image guided surgery system having i) a digitizer for tracking the position of the instrument in three dimensional space and ii) a display providing an indication of the position of the instrument with respect to images of a body part taken preoperatively. Preferably, the computer tracks the trajectory of the tool and the depth of the instrument inserted into the body part. In some embodiments, the computer-assisted image guided surgery system is that disclosed in U.S. Pat. Nos. 6,021,343; 5,769,861 & 6,428,547, the specifications of which are incorporated by reference.

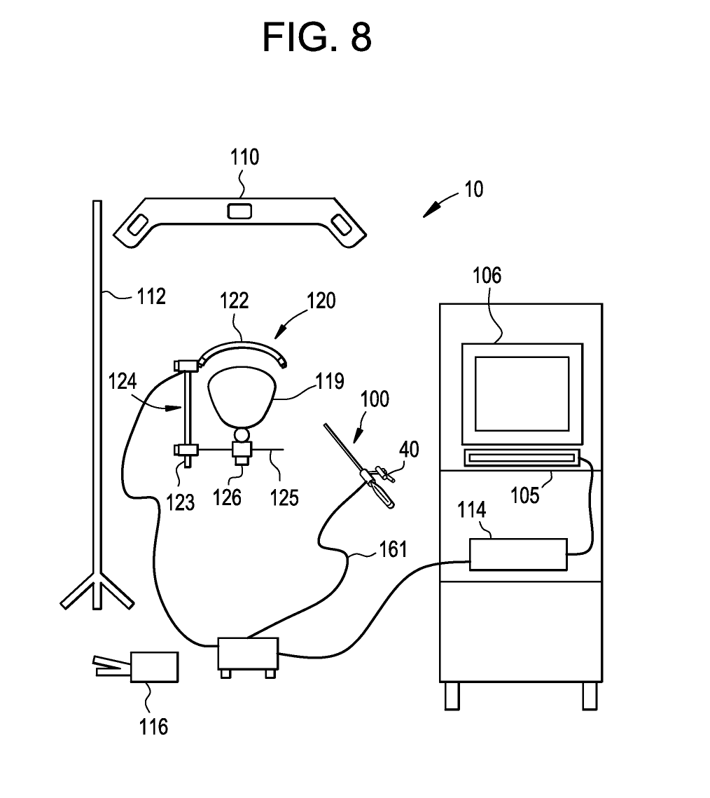

The medical instrument of the present invention is shown generally at 10 in FIG. 8. Instrument 100 can be used in many known computer assisted image guided surgical navigation systems and disclosed in PCT Publication No. WO 96/11624, incorporated herein by reference. A computer assisted image guided surgery system, shown at 10, generates an image for display on a monitor 106 representing the real time position of a body part (such as a spine) and the contoured virtual rod relative to the body part. Imaging of the spine may be carried out by intraoperative imaging such as a fluoroscope or intraoperative CT or preoperative imaging from a CT. In some embodiments, the surgeon may desire real time positioning of the spine. An image may be generated on touch screen 106 from an image data set stored in a controller, such as computer 108, usually generated preoperatively by some scanning technique such as by a CAT scanner or by magnetic resonance imaging. The image data set and the image generated have reference points for at least one body part. The reference points for the particularly body part have a fixed spatial relation to the particular body part.

System 10 also generally includes a processor for processing image data, shown as digitizer control unit 114. Digitizer control unit 114 is connected to monitor 106, under control of computer 108, and to instrument 100. Digitizer 114, in conjunction with a reference frame arc 120 and a sensor array 110 or other known position sensing unit, tracks the real time position of a body part, such as a cranium shown at 119 clamped in reference frame 120, and an instrument 100. Reference frame 120 has emitters 122 or other tracking means that generate signals representing the position of the various body reference points. Reference frame 120 is fixed spatially in relation to a body part by a clamp assembly indicated generally at 124,125, and 126. Instrument 100 also has a tracking device shown as an emitter array 40 which generates signals representing the position of the instrument during the procedure.

Sensor array 110, mounted on support 112, receives and triangulates the signals generated by emitters 122 and emitter array 40 in order to identify during the procedure the relative position of each of the reference points and the tip of the tracking device. Digitizer 114 and computer 108 may then modify the image date set according to the identified relative position of each of the reference points during the procedure. Computer 108 may then generate an image data set representing the position of the body elements and the virtual rod during the procedure. System 10 may also include a foot switch 116 connected to instrument 100 and digitizer 114 for controlling operation of the system. The structure and operation of an image guided surgery system is well known in the art and need not be discussed further here.

When the above is combined with the ability to capture intraoperative positions of the spine, the system could be used to capture the final spinal position and relate it to the virtual condition. It could relate, for example, that 90% of the planned sagittal correction has been achieved.

One skilled in the art will appreciate that the rods manipulated in the methods of the present invention may be configured for use with any type of bone anchor, e.g., bone screw or hook; mono-axial or polyaxial. Typically, a bone anchor assembly includes a bone screw, such as a pedicle screw, having a proximal head and a distal bone-engaging portion, which may be an externally threaded screw shank. The bone screw assembly may also have a receiving member that is configured to receive and couple a spinal fixation element, such as a spinal rod or spinal plate, to the bone anchor assembly.

The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, the specification of which is incorporated herein by reference in its entirety. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, the specifications of which are incorporated herein by reference in their entireties.

In some embodiments, the assembly may be implanted in accordance with the minimally invasive techniques and instruments disclosed in U.S. Pat. No. 7,179,261; and U.S. Patent Publication Nos. US2005/0131421; US2005/0131422; US 2005/0215999; US2006/0149291; US2005/0154389; US2007/0233097; and US2005/0192589, the specifications of which are hereby incorporated by reference in their entireties.

I claim:

1. A method comprising the steps of:
a) implanting a plurality of pedicle screws into the spine of a patient, each screw having a head,
b) contacting a tracking device to each head to allow a computer system to construct a virtual rod therefrom,
c) reading a geometric descriptor of the virtual rod displayed by the computer system.

2. The method of claim 1 further comprising the step of:
d) cutting a length of a rod blank based upon the geometric descriptor of the virtual rod.

3. The method of claim 1 further comprising the step of:
d) altering a contour of virtual rod.

4. The method of claim 1 further comprising the step of:
d) altering a contour of a physical rod based upon an image of the virtual rod projected onto a surface.

5. The method of claim 1 wherein the coupling step includes attaching

[symbol]

6. The method of claim 1 wherein the image of the virtual rod is a coronal or saggital image

7. The method of claim 1 wherein the geometric descriptor is a length of the virtual rod.

8. The method of claim 1 wherein the geometric descriptor is an image of the virtual rod.

9. The method of claim 1 further comprising the step of:
d) touching a computer touch screen to effect alteration of a contour of virtual rod.

10. The method of claim 1 wherein the geometric descriptor is a 2D image of the virtual rod in the coronal or sagittal plane.

11. A method comprising the steps of:
a) identifying locations of a plurality of screw heads attached to the spine of a patient,
b) creating a virtual rod from the locations of the screw heads.

12. The method of claim 11 wherein the locations of the screw heads are identified by locating a tracking device attached to each screw head.

13. The method of claim 12 wherein the virtual rod is created by a best fit line of the screw head locations.

14. The method of claim 11 further comprising the step of:
c) communicating a geometric descriptor of the virtual rod.

15. The method of claim 14 wherein the geometric descriptor is a length of the virtual rod.

16. The method of claim 14 wherein the geometric descriptor is an image of the. virtual rod.

17. The method of claim 14 wherein the image of the virtual rod is displayed on a surface.

18. The method of claim 11 further comprising the step of:
c) providing an image of the virtual rod.

19. The method of claim 11 further comprising the step of:
c) providing an image of an altered virtual rod.

20. The method of claim 11 wherein the altered virtual rod is based upon surgeon alteration of the virtual rod.

21. A computer comprising:
a) means for identifying locations of a plurality of screw heads attached to the spine of a patient,
b) means for creating a virtual rod from the locations of the screw heads.

22. A method comprising the steps of:
a) implanting a plurality of implants into the spine of a patient,
b) coupling a tracking device to each implant to allow a computer system to construct a virtual rod therefrom,
c) reading a geometric descriptor of the virtual rod displayed by the computer system.

23. The method of claim 21 wherein the implants are threaded implants.

24. The method of claim 21 wherein the coupling includes attaching.
